(19) United States
(12) Patent Application Publication
(10) Pub. No.: US 2016/0041162 A1
(43) Pub. Date: Feb. 11, 2016

(54) Novel Immunotherapy Libraries

(71) Applicant: Allan Yang Wu, Cathedral City, CA (US)

(72) Inventor: Allan Yang Wu, Cathedral City, CA (US)

(21) Appl. No.: 14/457,059

(22) Filed: Aug. 11, 2014

(51) Int. Cl. G01N 33/554; C07K 16/00; A61K 35/14

(52) U.S. Cl. CPC G01N 33/554; A61K 35/17; C07K 16/00; C07K 2317/14; C07K 2317/21; C07K 2317/90; G01N 2500/04; G01N 2500/10; G01N 2333/46

(57) ABSTRACT

Following a novel immunotherapy process the archived aliquots of biologic material from each successive process is used to form a library in which further novel therapy can be developed. The patients essentially serve as the biologic assay for successful antigen processing response in the form of clinical response (i.e. tumor shrinkage, improvement in clinical status of disease). Only archived biologic material of clinically successful patients are then further used for further drug development. These patients are also evaluated for a positive MCH I or MHC II response. In the case of MHC II or antibody response: antibody producing cells (B-cells) are harvested or biopsied from various B-cell containing tissues and fused with multiple myeloma cells to form a further antibody production library, which may be further screened for antibodies to relevant antigens. In the case of MHC I or cell-mediated cytotoxic T or NK cell immune response, the archived electroporated immune cells can propagated and expanded in culture and transplanted back to the original patient or other patients in an allogeneic fashion.

### CROSS REFERENCE TO RELATED APPLICATIONS

This invention claims benefit of priority of U.S. provisional patent application Ser. No. 61/959,045 filed Aug. 13, 2013.

### BACKGROUND

A novel immunotherapy process, whereby novel immunologic cells (non-dendritic) electroporated with novel antigens and transplanted back to the patient, has been detailed in a previous patent. Aliquots of the cellular and biologically derived materials during each phase of the immunotherapy process may be preserved and archived (via cryopreservation, refrigeration or lyophilization) to create “libraries” for further interrogation and drug or therapy development.

Following successful completion of the above immunotherapy method, further library screening for additional autologous or allogeneic or xenogenic therapy can be done if the subject achieves a therapeutic result. A therapeutic effect would be evidenced by either the presence of an antibody response to the antigens (as in the case of MHC II presentation) and/or a cytotoxic response (MHC I pathway). The purpose of this patent then is to present a novel method of library screening for the development of immunotherapeutics. The key advantage of this method is that a documented clinical response is known to occur and drug development can in fact be “reverse engineered” either by way of creating a fully humanized antibody or propagating previously archived electroporated immune cells.

### SUMMARY OF THE INVENTION

The invention is a method of screening treated subjects (using novel immunotherapy)for a therapeutic response based on clinical assessment (i.e. CT, MRI, tumor markers, surgical evaluation) and then further screening them for MHC I or MHC II responses. Archived aliquots such as the lysate antigens and electroporated immune cells (used in the initiating the immunotherapy process) may be used for additional future therapy.

For example, an individual that achieves a therapeutic response may have developed antibodies to the electroporated (or cultured) antigens. This may be tested by taking the original antigen lysate and using a dot blot or “atlas” like method to screen serum of successfully treated patients. A positive antibody response would warrant the harvest of immune tissues (i.e. lymph nodes, marrow, spleen, adipose tissue, omentum) to isolate antibody producing cells. The antibody producing cells may then be fused with multiple myeloma cell lines to create a human-human hybridoma library to further screen for clones strongly positive for antibodies directed against antigens originally used to innoculate the immune cells used to generate an immune response. In this way a fully humanized therapeutic monoclonal antibody may be further mass produced for both allogeneic and autologous therapy.

In another example a patient might achieve a therapeutic response with an MHC I, cytotoxic NK-cell mediated pathway activation, in which case only a clinical response WITHOUT a detectable relevant antibody would be appreciated. In this case, the archived electroporated immune cells may be propagated in culture to be given to the original donor or in an allogeneic fashion to a different individual. This may be possible in the case of mesenchymal stem cells which not only have the uncanny ability to perform antigen presentation, but also have the ability to escape immune system detection (self vs. non-self) and therefore can be transplanted allogenically.

This then allows for a “library” or archival method of further therapy of drug development, whereby the typical therapeutic antibody production or cellular therapy product is in a sense “reverse engineered” based upon a verifiable clinical response.

### BRIEF DESCRIPTION OF DRAWINGS

FIG. 1: MHC I pathway library screening and therapy derivation.

FIG. 2: MHC II pathway library screening and therapy derivation.

### DETAILED DESCRIPTION OF DRAWINGS

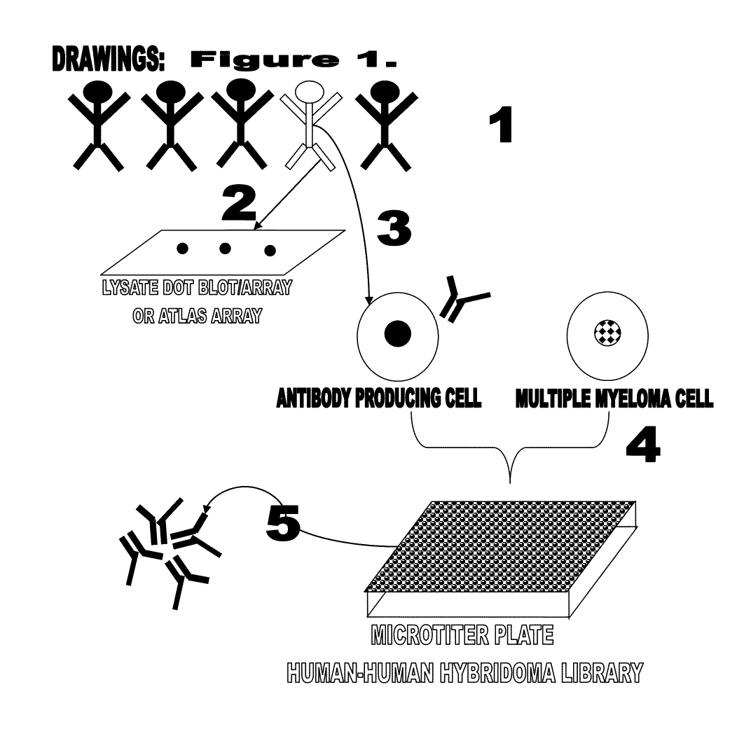

FIG. 1: MHC I pathway library screening and therapy derivation. Patients are initially screened for a positive clinical response (1), then their serum is screened for a positive MHC II response by detecting positivity (noted as a black filled in circle) on a dot blot or atlas array which may be made of original whole cell lysate used originally to mount an immune response (2). The patient is then biopsied/harvested of immune cell bearing tissues to obtain antibody producing cells and electro-fused with human myeloma cells to create hybridoma (3), several of these cell pairs or hybridomas may be created to ultimately form a searchable library on a titer plate (4) for production of relevant antibodies (5) which may ultimately be used for therapeutic or diagnostic reasons or even used in an allogeneic or xenogeneic fashion.

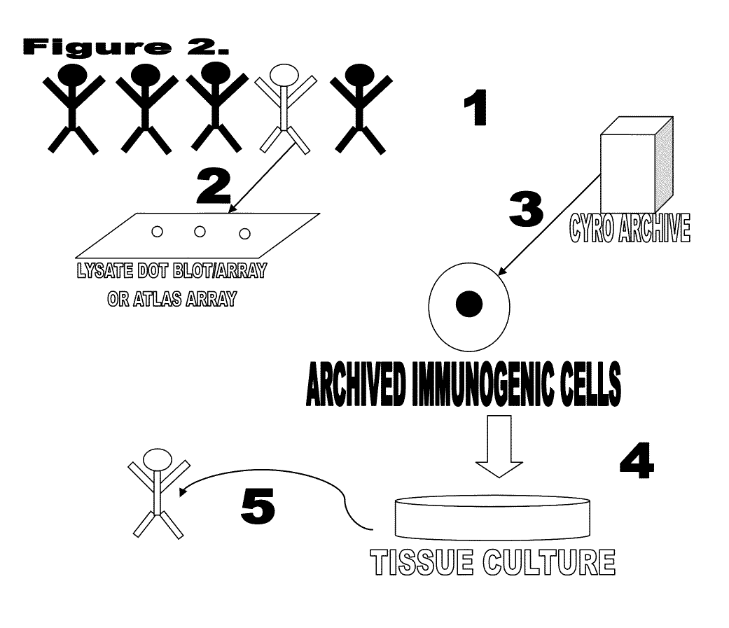

FIG. 2: MHC II pathway library screening and therapy derivation. Patients are initially screened for a positive clinical response (1), then their serum is screened for a negative MHC I response by detecting no binding (noted as an open white circle) on a dot blot or atlas array which may be made of original whole cell lysate used originally to mount an immune response (2). The cryo-archived immunogenic cells (either electroporated previously with antigen or un-electroporated) are thawed (3), the cells are further propagated in culture (4) and transferred back to the original host (5) or another human, plant or animal.

I claim:

1. A method of creating and screening libraries of archived biological material from a novel immunotherapy for further developing drugs and additional immunotherapy wherein:
a. if a successful MHC I mediated response is detected in the host, archived immunotherapy cells are propagated in culture and given back to the patient or another human or animal and/or
b. if a successful MHC II mediated response is detected in the host, antibody cell producing tissue is biopsied/harvested from the patient and the antibody producing cells are then fused to multiple myeloma cells to create a human-human hybridoma library
i. these hybridoma cell libraries may be interrogated for binding activity against relevant antigens
ii. the fully human antibodies of utility derived from the library may be used in any human or animal for therapeutic, research or diagnostic purposes
iii. a successful MHC II response (before library creation) may be discovered by using the archived whole cell lysate (antigens) in the form of a dot blot atlas to detect antibodies from serum of patients previously treated with novel immunotherapy.
